SEQUENCE LISTING (9 sequences)

SEQ ID NO: 1 - DNA, 1333 bp, Polypedilum vanderplanki
  source 1..1333: mol_type = genomic DNA
cttcaattatgatacatgaataaacaaaatattaaagaataaaattgtttttcttcattcatattgaatttttatttattattttgttaacttttgtcttttaaaaatttcaaagatatttcataaattcatctgaaattctcagatttataatgtaatgttgaaaaaatcgtatttatttttttgcgtaaatgaaagggaaattgatttcgcaatccaaaagaagaatttcttttacattttgtatttaaagatacgcaaaaaagaacacaaattcataataattttcaaagtcaacagaagtacattgcttctataaaaattatttaaaatttaaaatttgtgaaaagaattttaaaatgtgtggtagaaataaaaatcttaataaaagtcagtgcgagtgtcagcatcatcatcatcatcatttttatatcaatgaacagttaatagaacctgaggaaaaatcatcaaaattcaaatgattcagcaaaccatcaatacatcactatttttacttcaacgataagcaaataaataaagaagattgtcagccgaatgttgctacactcgcgaaaaatacagtaaaagaacatttcattcaacatcaaaaagctgcatctcaagaaaaaaattcaggctgctatcactttgaacataatccaattgataagaaatgggcacagtattttgaagagagatttggaaaatcacatggacatcaccattatcattcgttcaataatgatgattttccaaaaatgtttttcaacaaaaaatattttgaaggaagtttccttgattaagcaattcatatatttgaaagagaatttttgcatttgatttttttttcataaaaattaatttgtcgtttataaaacatatttgtcagtatgtttattttaaaaaatttaaatgagtaaaaaaactaatgaaaactataaataagtgaatttttatttcatttgtacaaacaatgattatcatcattatctcatcaatttcatcacttttagagaattttcacaaaaaaaaattatttttctacagaaaaatcacaaaataatgaaattcttatcaatataaaaaatgcatgtcattaccggcaattactcatatacaaaacaaacaagcacaaacacgttcaaaatcatattttcctcactatacaatataattattgtacacatgctcatttatttcacaagaattgtaaagaatattgtgagtgtatgtgaagcatataaaaagcagattaaatcgtacaacattcagttgacttatgatttctaaacgaataacatcgttggacaaaagaaaatattttctgaaaaaa

SEQ ID NO: 2 - DNA, 1353 bp, synthetic construct
  misc_feature 1..1353: note = artificial sequence
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggaattctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtaa

SEQ ID NO: 3 - DNA, 1461 bp, Drosophila melanogaster
  source 1..1461: mol_type = genomic DNA
atgacaacctcgatgcagccgagcaagtacacgggcctggtcgccgacctgatgcccaacatccgggcgatgaagtactccggcctgttcatgcacaacttcacgggcggcagtgccttcatgaagaaggtgtactcctccgtgcacctggtgttcctcctcatgcagttcaccttcatcctggtcaacatggccctgaacgccgaggaggtcaacgagctgtcgggcaacacgatcacgaccctcttcttcacccactgcatcacgaagtttatctacctggctgttaaccagaagaatttctacagaacattgaatatatggaaccaggtgaacacgcatcccttgttcgccgagtcggatgctcgttaccattcgatcgcactggcgaagatgaggaagctgttctttctggtgatgctgaccacagtcgcctcggccaccgcctggaccacgatcaccttctttggcgacagcgtaaaaatggtggtggaccatgagacgaactccagcatcccggtggagataccccggctgccgattaagtccttctacccgtggaacgccagccacggcatgttctacatgatcagctttgcctttcagatctactacgtgctcttctcgatgatccactccaatctatgcgacgtgatgttctgctcttggctgatattcgcctgcgagcagctgcagcacttgaagggcatcatgaagccgctgatggagctgtccgcctcgctggacacctacaggcccaactcggcggccctcttcaggtccctgtcggccaactccaagtcggagctaattcataatgaagaaaaggatcccggcaccgacatggacatgtcgggcatctacagctcgaaagcggattggggcgctcagtttcgagcaccctcgacactgcagtcctttggcgggaacgggggcggaggcaacgggttggtgaacggcgctaatcccaacgggctgaccaaaaagcaggagatgatggtgcgcagtgccatcaagtactgggtcgagcggcacaagcacgtggtgcgactggtggctgccatcggcgatacttacggagccgccctcctcctccacatgctgacctcgaccatcaagctgaccctgctggcataccaggccaccaaaatcaacggagtgaatgtctacgccttcacagtcgtcggatacctaggatacgcgctggcccaggtgttccacttttgcatctttggcaatcgtctgattgaagagagttcatccgtcatggaggccgcctactcgtgccactggtacgatggctccgaggaggccaagaccttcgtccagatcgtgtgccagcagtgccagaaggcgatgagcatatcgggagcgaaattcttcaccgtctccctggatttgtttgcttcggttctgggtgccgtcgtcacctactttatggtgctggtgcagctcaagtaa

SEQ ID NO: 4 - DNA, 1158 bp, Drosophila melanogaster
  source 1..1158: mol_type = genomic DNA
atggacagttttctgcaagtacagaagagcaccattgctcttctgggctttgatctctttagtgaaaatcgagaaatgtggaaacgcccctatagagcaatgaatgtgtttagcatagctgccatttttccctttatcctggcagctgtgctccataattggaagaatgtattgctgctggccgatgccatggtggccctactaataaccattctgggcctattcaagttcagcatgatactgtacttacgtcgcgatttcaagcgactgattgataaatttcgtttgctcatgtcgaatgaggcggaacagggcgaggaatacgccgagattctcaacgcagcaaacaagcaggatcaacgaatgtgcactctgtttaggacctgtttcctcctcgcctgggccttgaatagtgttctgcccctcgtgagaatgggtctcagctattggttagcaggtcatgcagagcccgagttgccttttccctgtctttttccctggaatatccacatcattcgcaattatgttttgagctttatctggagcgctttcgcctcgacaggtgtggttttacctgctgtcagcttggataccatattctgttccttcaccagcaacctgtgcgccttcttcaaaattgcgcagtacaaggtggttagatttaagggcggatcccttaaagaatcacaggccacattgaacaaagtctttgccctgtaccagaccagcttggatatgtgcaacgatctgaatcagtgctaccagccgatcatctgcgcccagtttttcatttcatctctgcaactctgcatgctgggatatctgttctccattacttttgcccagacagagggcgtgtactatgcctcattcatagccaccatcattatacaagcctatatctactgctactgcggggagaacctgaagacggagagtgccagcttcgagtgggccatctacgacagtccgtggcacgagagtttgggtgctggtggagcctctacctcgatctgccgatccttgctgatcagcatgatgcgggctcatcggggattccgcattacgggatacttcttcgaggcaaacatggaggccttctcatcgattgtccgcacggcgatgtcctacattacgatgctgagatcattctcctaa

SEQ ID NO: 5 - DNA, 24 bp, synthetic construct
  misc_feature 1..24: note = Synthetic
  source 1..24: mol_type = other DNA
ggttcgagcggtggtagcagcggc

SEQ ID NO: 6 - DNA, 665 bp, Polypedilum vanderplanki
  source 1..665: mol_type = genomic DNA
cgtcagttgaaattcagatatgaaacagtcaatgcgcttttcggttataattaaaagtcaaaaataaaaagaactgagaagaaaaagaacagtttcaaagtcatttttattttattataatttattttattttttttcttgtgtttatgctttttaaaagccgatgatgcaaattggatagacaagagtgcaaaaaaattattatatgtgttaaactagtccaacacagtgagaaagttaacagtcaatcaatggggaagtgaaatctgcaaagaggcaaaaaaattaaaaaattatagaaacgctcttatgaccttgttgaaaaataattacaaattattataaatgtattatataattttatgaatgtcgaaaaattttaattttttatatgtattattctataaaaaagaataaaaattgcatagtatacaaaaaagtttaataaaaattttaaaaaaaggtgactaatatagtgaaaaaaaaaatctattaattcttttaatgtgtagcttgaaaaacgtgaacttttaatttataaggccagaacagaaaattattagcgagaaacaatttatttgaatttcattactgcctcgtcacataagtgagaaaatatttttatttttgtggactaaaattggtcctctaataacaatttcaaa

SEQ ID NO: 7 - DNA, 677 bp, Polypedilum vanderplanki
  source 1..677: mol_type = genomic DNA
tccataggacaaagtatacatactattttacgtgttttactttattttctcacacctccttatttttaactgacatcaagccaaaaaattatttttcaaatgaccatgcataataatgatgataatagatttttaggtagacgtagactgcagtaaaagtagaaatagacaatgattttaaaaaaggaaattgttttagatgaatttatatttgtatctgatttaatcagtttgattattgtgataaagagagaaaatttcaataatttggacatatgtttttcgaataagctggataatattgtgaaataattgcaagatgcaagatgcgaatggaaatttttttatcgttaaatagatatccacatataaattgctgagttcaaaattgaatctcaataattttgccataattttagttttaaaccatgttatttgattaatgcttatttgagtatagcctttataggcttttaaattagttgacatttgcaatataaacaagttagtttatgaatgatgatgtagatagtaaaggaacacatacactaatatttattacaacttcatttctggataaaaatttttcaagttccttttttcaataaaaattaatagtgtatcccatgacacaacttattttaatataacaataaaatttagaaacataaaacagt

SEQ ID NO: 8 - DNA, 226 bp, Polypedilum vanderplanki
  source 1..226: mol_type = genomic DNA
cagtctactctttgcagttgttcattcacattcgaacgattaaggttgacaaaaaaatatatacgataattaatttaaaatcttctaattgtaaaatcaaataaatgaataatattttatctataaaatcttgaaatttagaacaaaaaagaaatagtgcataaacttgtcagttaataaaatcaattcaaatttcatataagcaaagcaaaatagacaaatcaaa

SEQ ID NO: 9 - DNA, 849 bp, Polypedilum vanderplanki
  source 1..849: mol_type = genomic DNA
atttttatcaatatatttacattcacatgacacaatttagttcttaaaaaaatgcaaaaccaaattttattagattgctattgtcatgattttattttttattttattgttttttgttagtcaactcaatagtaattgcaatgtatgtagtaaattgttctgaaagattctaataattttaaaaaatggaatcatatcatgatgctgggaactctaatgtttaattacatctcttcataattattgttattttttttattaataatttatcattatccatgaatgatataaatttagataaagccaagcctatgtttgcgacttatcgtcatcacaaagtaataaaaaaattactgtctaatgatagaaagggtgaagcttttgctacctaatttttgattggtcgctcaataaaataaaagcactagattatcatacattgttaattgttcttaaatgtaattgtctgtcattgagtaaaatttgagcaattttaattattgggctgcattttgaagatattttatgtctatttatcgactgtgtgcccttaatgacatcaagaaatatactatttattcttcaaaaacatatacatgtattcattcatcgctatgtactaaatcaagtttttgaaacctaattcaaagttataaaaaattgtagacggtcttctaattaaatatgtattgcatgtgagaaaaaaaaaacaagcgggagaaaatattaggtttacaaaaaatggacaagtttgaacttgtgtaaaaacgcatagaagaataagttattataagctgaaaaactgatgtaaatatgtcaaaatctaataaaatacttatttacgaagta

(19) United States
(12) Patent Application Publication
(10) Pub. No.: US 2025/0197799 A1
(43) Pub. Date: Jun. 19, 2025

(54) CELLS DERIVED FROM POLYPEDILUM VANDERPLANKI AND ODOR SENSOR EQUIPPED THEREWITH

(71) Applicants: University of Maryland, College Park, College Park, MD (US); National Agriculture and Food Research Organization, IBARAKI (JP)

(72) Inventors: Elisabeth Smela, Silver Spring, MD (US); Pamela Ann Abshire, Silver Spring, MD (US); Hiroto Fuse, Ibaraki (JP); Yugo Miyata, Ibaraki (JP); Takahire Kikawada, Ibaraki (JP); Richard Cornette, Ibaraki (JP)

(73) Assignees: University of Maryland, College Park, College Park, MD (US); National Agriculture and Food Research Organization, IBARAKI (JP)

(21) Appl. No.: 18/845,167

(22) PCT Filed: Mar. 10, 2023
(86) PCT No.: PCT/US2023/014959

§ 371 Date: Sep. 9, 2024

Related U.S. Application Data
(60) Provisional application No. 63/269,167, filed on Mar. 10, 2022.

(30) Foreign Application Priority Data
Mar. 10, 2023 (JP) 2023-037739

(51) Int. Cl. C12N 5/07; C07K 14/72; G01N 33/50

(52) U.S. Cl. CPC C12N 5/0601; C07K 14/723; G01N 33/5041; G01N 2333/726

(57) ABSTRACT

An object of the present invention is to provide cells that express a membrane protein as an exogenous protein but can be stably transported without a complicated culture apparatus. Cells from Polypedilum vanderplanki expressing an exogenous membrane protein are able to stably transport the membrane protein without a complex culture apparatus.

### GOVERNMENT SUPPORT

This invention was made with government support under CBET1842315 awarded by the National Science Foundation (NSF). The government has certain rights in the invention.

This application claims benefit and priority to U.S. Provisional Application No. 63/269,167 filed on Mar. 10, 2022, which is incorporated herein by reference in its entirety.

### TECHNICAL FIELD

The present disclosure generally relates to cells derived from Polypedilum vanderplanki expressing an exogenous membrane protein, an odor sensor comprising cells expressing odorant receptors (ORs), and methods for detecting an odor using cells expressing ORs.

### BACKGROUND

Polypedilum vanderplanki is a drought-tolerant insect, and it is known that even if its larvae have almost completely lost the water in their bodies and become dehydrated, if they are returned to water, they will resume growth as if nothing had happened. Focusing on this characteristic, research on cultured cells derived from Polypedilum vanderplanki is also underway; for example, cultured cells Pv11 are known to survive and resume proliferation after rehydration even when stored dry at room temperature for over a year (K. Watanabe et al. Cryobiology 73 (2016) 93-98). It is also known that the Pv11 cells protect the activity of enzymes in the cytoplasm even after long-term drying (Kikuta et al. “Towards water-free biobanks: long-term dry-preservation at room temperature of drying sensitive enzyme luciferase in airdried insect cells” Scientific Reports, 2017 (Published online 26 Jul. 2017)).

Conventional techniques such as seedling culture, PCR, chromatography, and gene expression analysis are used to identify the origin of agricultural products and to monitor the state of seedling infection in the field of food hygiene. For even more sensitive and real-time analysis by detecting trace odors, since there is a need for an odor sensor that replaces the prior art, various odor sensors such as semiconductor gas sensors have been developed. However, in order to detect trace amounts of odors, it is still the present situation that there is no choice but to rely on the olfactory sense of animals (Tetsuo Nozawa, NIKKEI Electronics, pp. 60-69, 2015. 06 Nikkei Business Publications). Therefore, as a biodevice having an odor detection function equivalent to the olfactory sense of animals, devices are being developed wherein cells that detect specific odors are arranged in an array, the response signals of the cells to the odor are detected, amplified, integrated with AI, and the like, and the composition of the odor is displayed. As a device, an odor sensor has been developed wherein an olfactory receptor protein (Or), which is an insect membrane protein; olfactory receptor protein co-receptor (Orco), which together with Or constitutes a cation channel; and GCaMP (GFP-based Ca²⁺-calmodulin protein; Nature Biotechnology volume 19, pages 137-141 (2001)), which is a fluorescent marker protein that detects changes in intracellular calcium concentration, were expressed in insect culture cells Sf21, the cells are arranged in an array and a specific signal pattern is detected for a particular odorant (Japanese Patent Application Publication No. 2013-27376). In addition, the development of a CMOS sensor that can detect action potentials of olfactory sensory neurons that respond to odorants with high sensitivity is also underway (T. Datta-Chaudhuri et al./Sensors and Actuators B 235 (2016) 74-78). It is expected that the development of odor biosensors using cultured cells will continue to progress.

It has been pointed out that existing biosensors using cultured cells or living tissue require the provision of particular maintenance conditions, depending on the cell or tissue type, such as the provision of nutrients. To use the biosensor as an odor sensor in the field, it is advantageous to be able to transport the odor sensor and to be able to store it in an environment where the humidity and temperature are not controlled until it is actually used.

In order to use cells expressing membrane proteins immediately when needed, the extracellular domain, transmembrane domain, and cytoplasmic domain that constitute the membrane protein must be expressed and stored in a state that maintains their function; however, maintaining normal cells requires a complicated culture apparatus. Further, in general, membrane proteins, especially extracellular domains, are directly exposed to environmental stresses such as high/low temperature and dryness, so it is difficult to stably retain membrane proteins in cell membranes. Accordingly, it is an object of the present disclosure to provide cells that express a membrane protein as an exogenous protein, but which can be stably transported without the need for a complicated culture apparatus.

As described below, to solve the above problems, it was discovered that by expressing an exogenous membrane protein in cells derived from Polypedilum vanderplanki, the cells can be stored at room temperature after being dried, and the cells can be rehydrated and conveniently used, when necessary, for detection of odorants and odors.

### SUMMARY

The present disclosure relates to an OR-based sensor system for detection of one or more odorant molecules using cells derived from Polypedilum vanderplanki expressing at least one exogenous membrane protein, wherein the exogenous membrane protein, such as an olfactory receptor protein, functions even when the cells are dried and then rehydrated.

Accordingly, the present disclosure provides cells derived from Polypedilum vanderplanki expressing at least one exogenous membrane protein. In an embodiment, the cells are insect cells, such as for example, the insect cell line Pv11. In a non-limiting embodiment, the exogenous membrane protein comprises an olfactory receptor protein (“Or”) and/or an olfactory receptor co-receptor (“Orco”) protein. In one embodiment, expression of the exogenous membrane protein gene is operably linked to the 121 promoter.

As used herein, the term olfactory receptor (OR) refers to a receptor translocated to the cell membrane after expression that is capable of detecting an odorant. In insects, ORs consist of a tetramer of two heptahelical subunits: a highly variable Or subunit that confers odor specificity (“olfactory receptor protein”; “Or”) and a co-receptor subunit, Orco that is highly conserved across insect species [23]. This hetero-complex forms an ion channel that is gated by odorant binding, directly allowing Na⁺ and Ca²⁺ influx. As used herein, “odorant” comprises any molecule that activates an olfactory receptor. Such odorants are very diverse and include pleasant and unpleasant odorants. The molecule that elicits the greatest response from an OR is referred to as its cognate ligand.

The excellent performance of animal olfactory systems for odor identification is due to signaling by multiple ORs responding to a single ligand, allowing discrimination of an enormous number of chemicals by combinatorial coding. Olfactory sensory neurons (OSNs), found in the olfactory epithelium of the noses of mammals or in the antennae of insects, typically express only a single type of OR. Animals may have hundreds of OR types to allow response to odors. As used herein, an “odor” comprises a mixture of odorants. Scents such as from food or coffee are odors. In applications outside of the laboratory, odorants are also found as components of odors since they are present in a background environment of other odorants.

ORs bind not just their cognate ligands, but other odorants also; an OR is considered broadly tuned if it binds a large number of odorants, and narrowly tuned if it binds a small number. Binding may lead not only to activation or excitation of the OR, but also to inhibition. Responses to mixtures are not simply linear and additive: the presence of a second odorant, even if it does not activate an OR, can modulate the response of the OR to another odorant.

A large number of ORs and combinatorial coding allows olfactory systems to respond to odors beyond their natural stimuli. For example, insects can distinguish explosives, drugs, and breast cancer. Biologically relevant concentrations cover a large range. Increasing odor concentration activates more OR types. ORs bind chemicals in a concentration-dependent manner and exhibit different affinities, and in general insect ORs are broadly tuned although they are most sensitive to structurally similar odorants (E. A. Hallem and J. R. Carlson, “Coding of odors by a receptor repertoire,” Cell, 125 (1), 143-160 (2006), 10.1016/j.cell.2006.01.050). The recruitment, with increasing concentration, of broadly tuned ORs that have lower affinity is an important principle that extends the dynamic range of the olfactory system.

An advantage associated with the use of Pv11 cells is that said cells are cultured at room temperature without special atmospheric conditions (e.g., CO₂). This contrasts with mammalian cells that require culturing at 37° C. at a particular pH, making Pv11 cells an excellent choice for sensors. In the dry state, they can be stored at varying temperatures, including room temperature, for prolonged periods of time after which they may be cultured. This unique feature for the biosensors described herein allows the sensors to be treated similarly to those using conventional polymer sensors. Upon rehydration a significant fraction of the OR protein-expressing cells resume function and proliferation, even after storage at room temperature for 3 years.

Further, as disclosed herein, the functions of multiple exogenous proteins are maintained after rehydration. Thus, in the cell line that stably expresses Orco-DmOr47a, the response to odorant resumes in less than an hour after rehydration. The response of the cells to odorants is enhanced further over the next 24 hrs by de novo synthesis of Orco and Or. In yet another embodiment, the engineered insect cells may express one or more additional exogenous proteins. “Exogenous protein” as used herein refers to a protein not naturally present in a particular organism, tissue or cell, for example, a protein from a different species. An “exogenous protein” may be a protein that is the expression product of an exogenous expression construct or transgene, or a protein not naturally present in a given quantity in a particular tissue or cell.

Such exogenous proteins may comprise a marker protein that detects activation of the olfactory receptor protein expressed within the cell. Such activation may result in changes in intracellular calcium concentration. Accordingly, in one embodiment the marker protein may include, for example, a fluorescent marker protein, e.g. a fluorescent calcium-sensing molecule. Cells having an activated OR will fluoresce and may be detectable by reading in an appropriate machine, such as a luminometer or fluorometer.

The present disclosure further provides an odor biosensor, comprising cells derived from Polypedilum vanderplanki expressing an Or and/or Orco. In a non-limiting embodiment, the Or proteins are derived from insect cells. In an embodiment, the odor biosensor cells are derived from Polypedilum vanderplanki and express the Or and/or Orco. The cells may further express a fluorescent marker protein that detects changes in intracellular calcium concentration. In one embodiment, the odor sensor cells are immobilized on a chip.

The present disclosure further provides an odor detection method comprising the steps of (a) providing biosensor cells expressing at least one Or and/or Orco and exposing the biosensor cells to at least one test compound or sample and (b) detecting a signal that indicates activation of the one or more ORs in the biosensor cells. In an embodiment, the detected signal is a change in intracellular calcium concentrations. In an embodiment, the change in intracellular calcium concentration is detected through the use of a marker protein that detects changes in intracellular calcium concentration, e.g., a fluorescent calcium-sensing molecule. In some embodiments, the biosensor cells expressing one or more ORs are placed in an assay plate, such as a 96-well plate or similar type plate and exposed to a test sample. In one embodiment, the odor sensor cells are immobilized on a substrate, such as a glass slide or CMOS chip. Further, multi-OR sensor arrays may be utilized to determine responses to odorants singly or in mixtures.

The biosensors provided herein may be used to detect odors from industry and livestock that can be detrimental to workers and nearby populations. Additionally, given that agricultural pests and affected plants release unique odors, the biosensors can be used to identify compounds for use in pest management. Biosensors may also be utilized for food safety; non-invasive medical diagnostics and health monitoring; detecting explosives and illicit drugs; detecting food spoilage; used in the production and authentication of perfumes, food products, beverages, wines, beer, and the like, detection of counterfeit food and perfume products; and used in search and rescue operations. In a specific embodiment, the biosensors may be used in the production and/or authentication of chocolate, wine, and olive oil. Further, there are many diseases transmitted by insect vectors, including sleeping sickness, river blindness, and Chagas disease. For these and other vector insects, olfactory cues are known to play a role in locating hosts and in other critical aspects of the life cycle. Accordingly, the biosensors disclosed herein may be used to identify compounds for use in control of diseases transmitted by insect vectors.

In certain embodiments, the present disclosure provides devices for detecting the presence of a particular odorant or for identifying an odor comprising biosensor cells expressing one or more ORs that are activated upon exposure to particular odorant molecules. In some embodiments the device is a handheld device. In some embodiments, the device is capable of communicating to a user of the device that a specific odorant has been detected or that an odor has been identified.

The present disclosure also provides kits for detection of an odorant or identification of an odor using one or more biosensor cells. In some embodiments, the kits comprise a panel of heterologous cells each expressing a particular OR. The kits may include instructions for using the kit. In other embodiments, the kits may also comprise buffers, and signal producing and detection systems.

A method of preserving an exogenous membrane protein is provided, the method comprising: a step of introducing a gene encoding the exogenous membrane protein into a cell derived from Polypedilum vanderplanki, a step of culturing the cell to express the exogenous membrane protein in the cell membrane, and a step of drying the cells. The method may further comprise, prior to the drying step, a step of suspending the cells in a solution comprising a dry protection agent.

According to the present disclosure, Polypedilum vanderplanki-derived cells can be provided that express exogenous membrane proteins that can be stored at room temperature after drying, and that can be rehydrated and conveniently used when necessary. Therefore, for example, if Or and Orco are employed as the exogenous membrane proteins, a portable odor biosensor can be produced, and it can be used for food hygiene control and food quality evaluation.

### BRIEF DESCRIPTION OF THE DRAWINGS

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1 is a schematic diagram showing one aspect of a cell according to the present disclosure.

FIG. 2 is a schematic diagram of the gene organization in the generation of Pv11-GCaMP6 f-Orco-OR47a stable expression cell line (Orco OR47a stable expression cell line). The lightning bolt symbol schematically indicates the three-salt front of the PAM sequence (NGG), which is the cleavage site of the CRISPR Cas9 protein.

FIG. 3 is a graph showing fluorescence intensity when a cell line stable expression Orco-OR47a is brought into contact with VUAA1 (N-(4-ethylphenyl)-2-{[4-ethyl-5-(pyridin-3-yl)-4H-1,2,4-triazol-3-yl]sulfanyl}acetamide), an agonist of Orco.

FIG. 4 is a graph showing the fluorescence intensity when a cell line stable expression Orco-OR47a is brought into contact with pentyl acetate, which is a ligand of OR47a (ns: non-significant, ***: P<0.0001).

FIG. 5 is a graph showing the viability 1 hour after drying and rehydrating Pv11 wild type (Pv11), Pv11-GCaMP6 f-Orco stable expression cell line (Orco stable expression cell line) and Orco-OR47a stable expression cell line.

FIG. 6A-B. FIG. 6A is a graph showing the response function to VUAA1, pentyl acetate, and acetophenone of the Orco-OR47a stable expression cell line as a change in fluorescence intensity before dehydration treatment (**:p<0.01, ***:p<0.001). FIG. 6B is a graph showing the response function to VUAA1 and pentyl acetate of the Orco-OR47a stable expression cell line as a change in fluorescence intensity after 1 hour of rehydration after drying (***:p<0.001, ****p<0.0001).

FIG. 7A-D. FIG. 7A. is a graph showing the response function to VUAA1 of the Orco-OR47a stable expression cell line as a change in fluorescence intensity after 1 hour of drying treatment. By treatment with CHX (protein synthesis inhibitor), membrane proteins by de novo protein synthesis were assumed to be inhibited (ns: non-significant). FIG. 7B is a graph showing the response function to pentyl acetate of the Orco-OR47a stable expression cell line as a change in fluorescence intensity after 1 hour of drying and rehydration. It was treated with CHX (protein synthesis inhibitor) as in FIG. 7A (ns: non-significant). FIG. 7C is a graph showing the response function to VUAA1 of the Orco-OR47a stable expression cell line as a change in fluorescence intensity after 24 hours of drying and rehydration. It was treated with CHX (protein synthesis inhibitor) as in FIG. 7A (****:p<0.0001). FIG. 7D is a graph showing the response function of the Orco-OR47a stable expression cell line 1 hour after rehydration and 24 hours after drying. It was treated with CHX (protein synthesis inhibitor) as in FIG. 7A (*** *:p<0.001).

FIG. 8 is a graph showing fluorescence intensity when VUAA1 or pentyl acetate was brought into contact with immobilized Orco-OR47a-expressing cell line (ns: non-significant, *:p<0.05, ****:p<0.01).

FIG. 9A-B. FIG. 9A is a graph showing the response function over time to pentyl acetate as a change in fluorescence intensity in a ligand assay utilizing a perfusion system using an Orco-OR47a stable expression cell line. FIG. 9B is a graph showing the response function over time to pentyl acetate as a change in fluorescence intensity in a ligand assay utilizing a perfusion system using a Pull-GCaMP6f stable expression cell line (GCaMP6f stable expression cell line).

FIG. 10 is a graph confirming that OR47a expression was optimized by adding an untranslated region (UTR) in a donor vector (*p<0.001, ****:p<0.0001).

FIG. 11A-E. Polypedium vanderplanki in adult FIG. 11A, Larval FIG. 11B and dried larval state FIG. 11C. The Oreo/OR ion channel opens upon odorant binding. Ca²⁺ influx FIG. 11D and leads to GCaMP reporter fluorescence FIG. 11E.

### DETAILED DESCRIPTION

The present disclosure relates to cells derived from Polypedilum vanderplanki expressing exogenous membrane proteins produced by genetic engineering techniques. Cells derived from Polypedilum vanderplanki for expressing the exogenous membrane protein can be those commonly used in the art without particular limitation; examples include Pv11 cells, Pv210 cells, and the like. Cells from Polypedilum vanderplanki expressing the exogenous membrane protein can be dried and then rehydrated to allow the exogenous membrane protein to function. Here, the drying conditions are not particularly limited as long as the exogenous membrane proteins are not denatured and the cells derived from Polypedilum vanderplanki can function by rehydration after drying; for example, they may be dried in an environment where the temperature is 30° C. or less, preferably 25° C. or less, and/or in an environment where the humidity is 10% or less, preferably 5% or less, for 2 days or more, preferably 7 days or more. Drying under such mild conditions allows the exogenous membrane proteins to function better after rehydration. In addition, during the drying, it is preferable from the viewpoint of guiding the expression of functions related to extreme drought resistance if the cells derived from Polypedilum vanderplanki are suspended in a solution containing a drying protective agent such as insect culture medium containing trehalose and serum and then dried.

Moreover, “rehydration” refers to the rehydration of dried cells into an aqueous solution. The conditions for the rehydration are not particularly limited as long as the dried cells derived from Polypedilum vanderplanki can function; for example, a buffer solution comprising an insect cell medium such as IPL-41 or Dulbecco's phosphate buffer (DPBS) may be added to the cells, and a solution comprising the insect cell medium is preferably added. The cells according to the present disclosure are characterized in that even a membrane protein having an extracellular domain such as a functional odorant receptor (heterocomplex) can be dry-preserved while maintaining its function. More than 20% of the dried cells survived after rehydration when stored at room temperature for 7 days or longer, and at least 1% survived after rehydration when stored at room temperature for 372 days or more.

In addition, the cells express newly produced exogenous membrane proteins in vivo by 24 hours after drying and rehydration; however, immediately after drying and rehydration, without waiting for the expression of the exogenous membrane protein produced in vivo, membrane proteins stored in a dried state begin to revive, and the state of cells can be stabilized in, for example, about 10 minutes. Moreover, even if the cells are dried again after the rehydration, the function of the membrane protein can be similarly preserved. For example, even if cells from Polypedilum vanderplanki expressing exogenous membrane proteins are dried, they can exhibit their functions immediately after rehydration.

The exogenous membrane protein is not particularly limited; examples include cell surface receptor proteins, channels, and transporters that respond to a wide variety of signals from the external environment such as chemical substances. Such cell surface receptor proteins include G protein-coupled receptor (GPCR) proteins, enzyme-coupled receptors, ion channel-coupled receptors, and the like. One aspect of the cell surface receptor protein includes, for example, olfactory receptor proteins (Or), olfactory receptor protein co-receptors (Orco), gustatory receptors with similar structure to Or and Orco, mechano-stimulatory receptors, and the like. In addition, the cell may express one type of exogenous membrane protein or may co-express two or more types of exogenous membrane proteins. Complexes may also be formed between co-expressed exogenous membrane proteins.

One aspect of the present disclosure is a cell from Polypedilum vanderplanki expressing olfactory receptor protein (Or) and/or insect olfactory receptor protein co-receptor (Orco). The olfactory receptor protein is a type of G protein-coupled ion channel in the olfactory receptor nerve; in the present disclosure, it may be derived from vertebrates such as mammals or from insects, but it is preferably derived from insects because it is simple because it functions with two proteins, Or and Orco, and because it is superior in diversity and odor selectivity.

More than 100 distinct insect olfactory receptor proteins from Drosophila melanogaster, Anopheles gambiae, Bombyx mori, and the like have been identified as described in Japanese Patent Application No. 2013-27376. Each OR has specific responses to odorants such as, for example, phenethyl alcohol, methyl benzoate, ethyl benzoate, benzyl alcohol, methyl salicylate, benzaldehyde, pentanal, hexanal, E2-hexanal, 2-heptanone, 6-methyl-5-hepten-2-one and 2-methylphenol (Hallem et al., Cell 125, 143-160, Apr. 7, 2006). The receptor protein functions as an ion channel-coupled receptor, and when the target odorant binds to the receptor, an influx of ions into the receptor-expressing cell occurs.

Insect ORs may be derived, for example, from the insect orders Coleoptera, Lepidoptera, Diptera, and Hymenoptera, including species of economic or medical importance. In an embodiment, the Or proteins for expression in Polypedilum vanderplanki cells are derived from Drosophila. Such Drosophila OR proteins include, but are not limited to, Or1a, Or2a, Or7a, Or9a, Or10a, Or13a, Or19b, Or19a, Or22a, Or22b, Or22c, Or23a, Or24a, Or30a, Or33a, Or33b, Or33c, Or35a, Or42a, Or42b, Or43a, Or43b, Or45a, Or45b, Or46a, Or47a, Or47b, Or49a, Or49b, Or56a, Or59a, Or59b, Or59c, Or63a, Or65a, Or65b, Or65c, Or67a, Or67b, Or67c, Or67d, Or69a, Or74a, Or82a, Or83a, Or83c, Or85a, Or85b, Or85c, Or85d, Or85e, Or85f, Or88a, Or92a, Or94a, Or94b, Or98a and Or98b.

In non-limiting embodiments, DmOr85b which responds to ammonia, classified as an extremely hazardous substance, DmOr46a which responds specifically to 4-methylphenol, present in human sweat, and DmOr98a which can detect phenyl-acetone, a precursor of amphetamines may be expressed in Polypedilum vanderplanki cells. The database DoOR2.0 includes findings on the function of ORs in Drosophila and quantitative data on odorant responses in vivo. (D. Münch and C. G. Galizia, “DoOR 2.0-Comprehensive mapping of Drosophila melanogaster odorant responses,” Sci. Rep., 6 (1), 21841 (2016), 10.1038/srep21841)

The olfactory receptor protein co-receptor (Orco) is a coupling factor with Or, and the ligand-binding Or and Orco form a heterocomplex and function as a ligand-gated ion channel. In general, when an odorant binds to the OR, the pore formed by the Or and Orco opens, allowing cations to flow into the cell and trigger an action potential. The insect olfactory receptor protein co-receptor (Orco) is highly conserved among species. Therefore, an Orco protein, not only derived from Drosophila melanogaster but also proteins suitable for Pv11 cells such as Polypedilum vanderplanki can be used.

In an embodiment, the cell derived from Polypedilum vanderplanki according to the present disclosure can have a gene encoding an exogenous membrane protein integrated into the genome of the cell, because the exogenous membrane protein can be stably expressed. In cells from Polypedilum vanderplanki expressing the exogenous membrane protein of the present disclosure, preferably, the gene encoding the exogenous membrane protein is operably linked to the 121 promoter (SEQ ID NO: 1). Here, the 121 promoter is a strong promoter discovered from the Polypedilum vanderplanki genome; it is known to have about 1,500-fold higher ability to produce protein compared to the promoter contained in commercial kits for insect cells. The 121 promoter derived from Polypedilum vanderplanki has the sequence shown in SEQ ID NO:1.

In another aspect, the Polypedilum vanderplanki-derived cells expressing the exogenous membrane protein of the present disclosure may further express another exogenous protein other than said exogenous membrane protein. The other exogenous protein is not particularly limited; for example, when the exogenous membrane protein is a G protein-coupled ion channel, a fluorescent marker protein capable of detecting ions flowing into cells when the ion channel binds to a ligand may be expressed as the exogenous protein. When the intracellular calcium ion changes as a result of the function of the exogenous membrane protein, the exogenous protein may be a protein that emits fluorescence in response to calcium ions in the cytoplasm, specifically, it may be, for example, G-CaMP; G-CaMP1. 6; GCaMP2; GCaMP3; G-CaMP4.1; GCaMP5; G-CaMP6, 7, 8; GCaMP6f, 6m, 6s; jGCaMP7f, 7s, 7b, 7c, GCaMP-X, and the like.

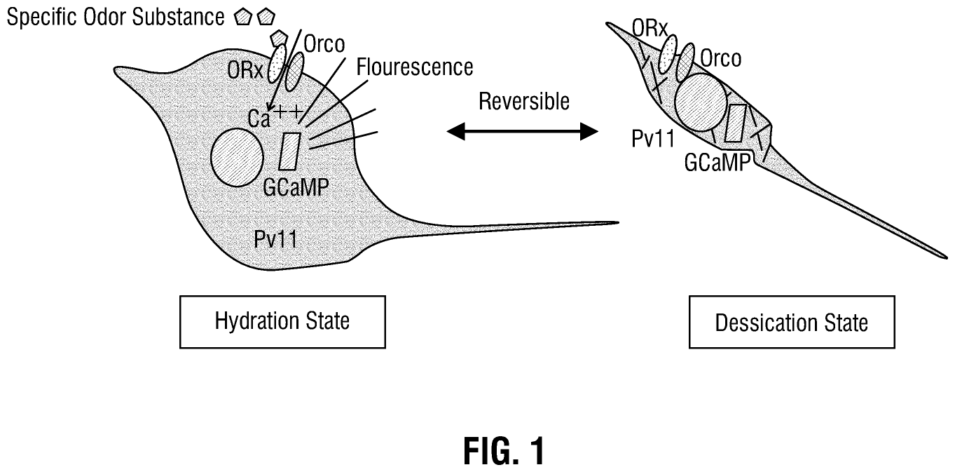

One embodiment of Polypedilum vanderplanki-derived cells according to the present disclosure is described with reference to FIG. 1. The cells of one embodiment are Pv11 cells co-expressing Or and Orco as exogenous membrane proteins and intracellularly expressing exogenous GCaMP. Even if the cells are dried, they can be restored to functional cells by rehydration, and the hydration and desiccation states are reversible. The dry cells can be stored at room temperature and can be transported without complicated culture equipment. Moreover, the exogenous membrane proteins can be functional even after drying and rehydrating the cells. That is, Or and Orco form an odorant receptor complex in the cell membrane, and when the complex comes into contact with an odorant that specifically reacts with it, the calcium ion concentration in the cytoplasm rises in response. Such responses can be reproduced in as little as one hour after drying and rehydrating the cells. Since GCaMP emits a fluorescent signal in response to calcium ions, it is possible to visualize an increase in calcium ion concentration due to the response reaction. Furthermore, 24 hours after rehydration, the odorant response of the cells can be potently enhanced by de novo synthesized Orco and Or proteins. Thus, if the Pv11 cells expressing Or and Orco and GCaMP remain hydrated even after repeated hydration and drying, because they can exert the function of the odorant receptor complex, which is a membrane protein, they can be shipped dry and rehydrated when needed to aid in the detection of particular odorants or the identification of odors.

For production of recombinant cells, a portion of the gene encoding an exogenous membrane protein can be obtained, for example, by extracting mRNA, synthesizing, and isolating cDNA, and using the cDNA as a template and appropriate PCR primers by PCR. Similarly, genes encoding additional exogenous proteins can be partially obtained by PCR using appropriate PCR primers using cDNA as a template. The exogenous membrane protein can be cloned by other conventional methods. The exogenous membrane protein and/or the additional exogenous protein can be incorporated into an expression vector, and cells derived from Polypedilum vanderplanki can be transformed with the expression vector to prepare the cells to express the protein of interest. Exogenous protein expression vectors are not particularly limited as long as they are replicable in host cells.

For expression of Or and/or Orco, recombinant DNA technology well known to those skilled in the art may be used. Expression vector may be a nucleic acid in the form of a plasmid, a cosmid, a phagemid, a phage, a viral vector or the like. For expression vector construction including recombinant DNA technology, reference may be made to Sambrook et al., Molecular Cloning, A Laboratory Manual, Cold Spring Harbor Laboratory Press, (2001), F M Ausubel et al, Current Protocols in Molecular Biology, John Wiley amp; Sons, Inc. (1994), and Marston, F (1987) DNA Cloning Techniques) and the like.

The expression vector may include regulatory sequences that affect transcription and translation of the Or and/or Orco encoding nucleic acid by being operably linked to the nucleic acid. Such regulatory sequences may include promoter sequences, transcription termination signal sequences (polyadenylation signal), and the like. As used herein, the term “being operably linked” means a linkage such that the transcription and/or translation of a nucleic acid are affected. Regulatory sequences also include enhancer sequences that function to regulate the transcription of a nucleic acid.

The expression vector may further include a selectable marker gene. The selectable marker gene is a gene encoding a trait that enables selection of a host microorganism containing such a marker gene and is generally an antibiotic resistance gene.

The expression vector may also include a restriction enzyme recognition site for easy cloning of the Or and/or Orco protein encoding nucleic acid. The expression vector may then be transformed into a host microorganism for expression of the Or and/or Orco protein.

In a specific embodiment, the strong promoter 121 may be used to express Ors and Orco in Pv11 cells.

Also, in order to obtain a stable expression strain that expresses the exogenous membrane protein and/or the additional exogenous protein, a known genome editing technique using ZFN, TALEN, CRISPR/Cas9, and the like may be applied to knock-in the gene encoding the exogenous membrane protein and/or the additional exogenous protein into Polypedilum vanderplanki-derived cells. In the present disclosure, among known genome editing techniques, it is preferable to use the CRIS-PITCh method (Naka de S et al., “Microhomology-mediated end-joining-dependent donor integration of DNA in cells and animals using TALENs and CRISPR/Cas9” Nature Communications 5:5560 2014) in knock in because of the ease of operation and the high accuracy of gene insertion. The CRIS-PITCh method is a gene knock-in method that introduces into cells a donor vector comprising a cassette in which a short homologous sequence (microhomology) of about 20 salt pairs is added to both ends of an exogenous gene, and utilizes the repair pathway MMEJ (non-homologous end joining) by cleaving both the genomic region of the target gene and the outer side of the exogenous gene at the same time. Further, sequences encoding a plurality of proteins of interest may be incorporated into one expression vector or donor vector, or sequences encoding each protein of interest may be incorporated into each vector. Expression of exogenous membrane proteins and/or additional exogenous proteins may also be optimized in the expression vector or donor vector. The optimization means is not particularly limited; for example, the addition of an untranslated region (UTR), codon optimization, addition of a cell membrane localization signal peptide, or the like can be employed. Preferably, expression of exogenous membrane proteins can be optimized by the addition of UTRs. Further, in cells derived from Polypedilum vanderplanki expressing the exogenous membrane protein and/or additional exogenous protein according to the present disclosure, the gene encoding the exogenous protein is preferably designed to be operably linked to the 121 promoter (SEQ ID NO:1).

The present disclosure also relates to an odor sensor comprising cells derived from Polypedilum vanderplanki, expressing one or more OR, preferably co-expressing Or and Orco. The cells may further express, as the additional exogenous protein, a fluorescent marker protein that detects changes in intracellular calcium concentration. Also, instead of having the cells express a fluorescent marker protein, a low-molecular-weight fluorescent substance such as calcium ion-dependent fluorescent dye for cells, for example, Calbryte™ 520 AM, Calbryte™ 590 AM, Calbryte™ 630 AM, Fluo-5F AM, Fluo-4 AM, Fluo-4FF AM, Fluo-3 AM, Fura-2 AM, Indo-1 AM, Rhod-2 AM, Rhod-3 AM, X-Rhod-1 AM, X-Rhod-5F AM, Oregon Green (registered trademark) 488 BAPTA series may be added to allow the intracellular calcium ion concentration to be observed by changes in fluorescence. In the odor sensor, the fluorescence intensity after contact with an odorant with which the OR specifically reacts preferably increases by at least 1% or more, preferably at least 5% or more, compared to the fluorescence intensity before contact.

In one aspect, the cells are immobilized on a chip. Although the means for fixing the cells is not particularly limited, the cells may, for example, be immobilized on the chip using a biocompatible anchor for cell membrane (BAM) or polyethylenimine, which is an immobilization system utilizing electrostatic interaction. Further, the odor sensor preferably comprises at least 1×10⁴ concentration/cm², preferably at least 1×10⁵ concentration of cells derived from Polypedilum vanderplanki, on the chip on which the cells are fixed. By providing a plurality of types of spots formed by fixing cells separately for each type of OR on the chip, an array capable of simultaneously detecting different types of odorants or for identifying odors can be produced. If the array is brought into contact with a test sample having an unknown composition, and the test sample contains an odorant to which any one of the ORs specifically responds, the fluorescence of spots where cells expressing the OR are fixed is observed. In addition, the odor sensor can be stored in a dry state and can be immediately used for odor determination by rehydrating the cells at the time of use. Methods commonly used in the art can be employed without particular limitation for the method of drying and rehydrating the cells, the method of detecting and analyzing fluorescence signals, and the like.

Accordingly, the present disclosure provides an odor detection method comprising the steps of (a) providing biosensor cells expressing at least one Or and/or Orco and exposing the biosensor cells to at least one test compound or sample and (b) detecting a signal that indicates activation of the one or more ORs in the biosensor cells. In an embodiment, the detected signal is a change in intracellular calcium concentrations. In an embodiment, the change in intracellular calcium concentration is detected through the use of a marker protein that detects changes in intracellular calcium concentration, e.g. a fluorescent calcium-sensing molecule. In some embodiments, the biosensor cells expressing one or more ORs are placed in an assay plate, such as a 96-well plate or similar type plate and exposed to a test sample. In one embodiment, the odor sensor cells are immobilized on a chip. Further, multi-OR sensor arrays may be utilized to determine responses to odorants singly or in mixtures.

The odor sensor can be applied, for example, to the management of prohibited goods at airports, the medical field aiming at early detection of cancer by odor, and the like. In the field of agriculture, it can also be applied to food hygiene control and food quality evaluation based on the detection of slight odors (for example, geosmin produced by mold). Another application is the early detection of agricultural pests by detecting specific patterns in an array of cells caused by an odorant or odor.

The disclosure also relates to a method of preserving exogenous membrane proteins in a dry state. The method comprises a step of introducing a gene encoding the exogenous membrane protein described above into cells derived from Polypedilum vanderplanki, a step of culturing the cells to express the exogenous membrane protein in the cell membrane, and a step of drying the cells. According to the method, immediately after drying and then rehydration of the cells expressing the exogenous membrane protein, the membrane protein stored in the dried state begins to revive, and the state of the cell can be stabilized in, for example, about 10 minutes. In addition, before the drying step, it is preferable to further comprise a step of suspending the cells in a solution comprising a drought-protecting agent such as insect culture medium comprising trehalose or serum from the viewpoint of induction of expression of functions related to extreme drought tolerance.

In certain embodiments, the present disclosure provides devices for detecting the presence of a particular odorant or for identifying an odor comprising biosensor cells expressing one or more ORs that are activated upon exposure to particular odorant molecules or to particular odorant mixtures. In some embodiments the device is a handheld device. In some embodiments, the device is capable of communicating to a user of the device that a particular odorant has been detected or that a type of odor has been identified.

The present disclosure also provides kits for detection of an odorant or identification of an odor using one or more biosensor cells. In some embodiments, the kits comprise a panel of heterologous cells each expressing a particular OR. The kits may include instructions for using the kit. In other embodiments the kits may also comprise buffers, and signal producing and detection systems.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The present disclosure will be described in more detail below with reference to embodiments, but the present disclosure is not limited to these embodiments.

### EXAMPLES

1. Transient expression strain preparation. GCaMP6f cDNA was amplified by PCR in a conventional manner, and a pPv121-GcaMP6f vector was produced by inserting the amplified DNA sequence (SEQ ID NO: 2) encoding GcaMP6f downstream of a 121 promoter of a pPv121-MCS vector (Tokumoto et. Al., doi: https://doi. Org/10.1101/2020.05.29.123570 “Development of a Tet-On inducible expression system for the anhydrobiotic cell line, Pv11”). Similarly, the cDNA of Orco derived from Drosophila melanogaster was amplified by PCR by a conventional method, and a pPv121-Orco vector was generated by inserting the amplified Orco-encoding DNA sequence (SEQ ID NO:3) downstream of the 121 promoter of the pPv121-MCS vector. In addition, Or47a cDNA derived from Drosophila melanogaster was amplified by PCR in a conventional manner, and the amplified DNA sequence encoding Or47a (SEQ ID NO: 4) was operably linked to pPv121-M.

The resulting pPv121-GcaMP6f vector, pPv121-Orco vector, and pPv121-Or47a vector were simultaneously transfected to Pv11 cells (see Miyata Y., et. Al, Scientific Reports, 9 (1), 7004. (2019)), and a Pv11 transient expression strain was produced that transiently expressed the GaMP6f, Orco, and Or47a proteins. If the membrane proteins Orco and Or47a were expressed and GcaMP6f was expressed in the cytoplasm, then by stimulating Pv11 transient expression strains with Orco or Or47a agonists or ligands, calcium ion influxed into the cells, and the fluorescence of GcaMP6f was observed. Therefore, in the medium comprising the obtained Pv11 transient expression strain, fluorescence was observed by adding dimethyl sulfoxide (DMSO; negative control), VUAA1 (Orco agonist), and pentyl acetate (ligand of Or47a) and photographing the cells under a fluorescence microscope. As a result, since fluorescence was observed both with VUAA1 stimulation and with pentyl acetate stimulation, it was confirmed that each exogenous protein was expressed in the obtained Pv11 cells. Therefore, it was confirmed that Pv11 cells are able to at least transiently express each exogenous membrane protein and that this is functional. SEQ ID NO: 2 represents GcaMP6f. SEQ ID NO: 3 represents Orco. SEQ ID NO: 4 represents Or47a.

2. Generation of cell lines with stable expression of exogenous membrane proteins and/or additional exogenous proteins.

2.1 Method for producing a cell line with stable expression of GCaMP6f. The Pv.00443 gene downstream of the 121 promoter, possessed by Pv11 cells, was used as the target gene. Just before the stop codon of the Pv.00443 gene, a gene fragment comprising the DNA sequence of GCaMP6f to which a P2A (self-cleaving 2A peptide) sequence was added was knocked in by the CRIS-PITCh method (Nakade S et al. “Microhomology-mediated end-joining-dependent donor integration of DNA in cells and animals using TALENs and CRISPR/Cas9” Nature Communications 5:5560 2014). In addition, in order to efficiently obtain knock-in transformants, a sequence encoding Zeocin-resistance gene (zeo′) to which a DNA sequence encoding P2A was added was also knocked in at the same time. Then, a cell line was established for stable expression of Pv11-GCaMP6f by performing selection with Zeocin and single-cell sorting using fluorescence of GCaMP6f.

2.2 Method for producing an Orco stable expression cell line. The method described in 2.1 above was used to create an Orco stable expression cell line. Just before the stop codon of the Pv.00443 gene downstream of the 121 promoter, knock in was performed with the DNA sequence of GCaMP6f to which the P2A sequence was added and the Orco DNA sequence to which the P2A sequence was added. An Orco-expressing cell line was established by single-cell sorting using GCaMP6f fluorescence.

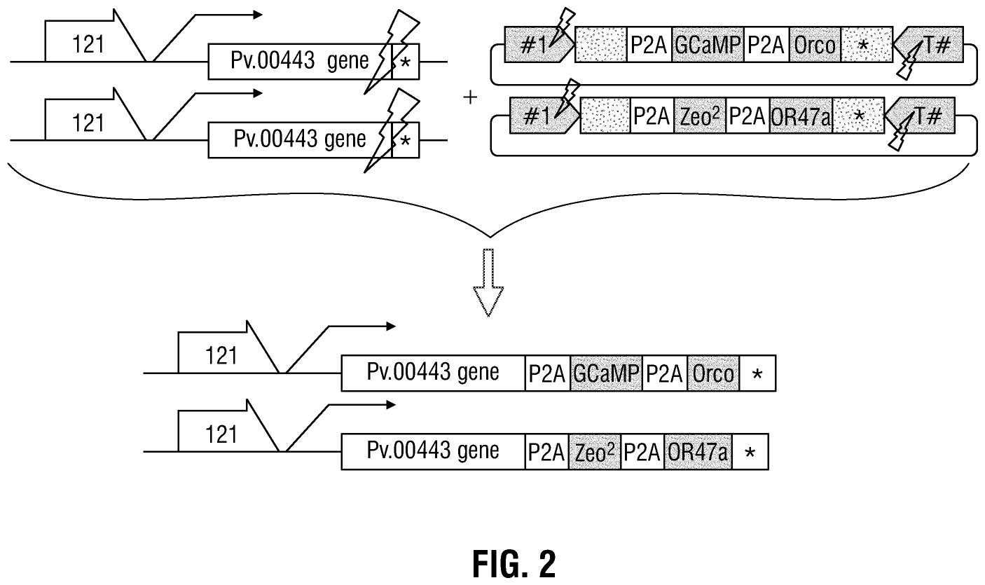

2.3 Method for generating Orco-Or47a stable expression cell line, The method described in 2.1 above was used to create an Orco-Or47a stable expression cell line as described above in 2.1 above. Just before the stop codon of the Pv. 00443 gene downstream of the 121 promoter, an exogenous gene fragment consisting of an exogenous gene fragment consisting of a GCaMP6f DNA sequence with a P2A sequence added; an Orco DNA sequence with a P2A sequence added; a zeo^{r} DNA sequence with a P2A sequence added; and a Or47a DNA sequence with a P2A sequence added was knocked in by the CRIS-PITCh method. An Orco-Or47a-expressing cell line was established by selection with Zeocin and single-cell sorting using GCaMP6f fluorescence. FIG. 2 schematically shows the gene organization in the preparation of the Orco-Or47a stable expression cell line. A lightning mark schematically indicates the cleavage site.

3. Evaluation of stable cell lines

3.1 Knock-in check. To confirm whether the target gene can be knocked into the target site in the established stable expression cell line, when genomic PCR was performed upstream and downstream of the Pv. 00443 gene, a band of the expected size was detectable. After gel purification of this band, DNA sequencing showed that the sequence matched the target (exogenous) gene. It was confirmed that the target gene was correctly knocked into the target site in each of the cell lines established above.

3.2 The functionality of GCaMP6f. The functionality of GCaMP6f in the resulting cell lines was evaluated using Ionomycin (Ionomycin; Fujifilm Wako Pure Chemical) to increase the intracellular concentration of calcium ions to provide functional confirmation of exogenous proteins in the GCaMP6f stable-expressing cell line. Ionomycin was dissolved in dimethyl sulfoxide (DMSO; Fujifilm Wako Pure Chemical Industries, Ltd.), adjusted to a final DMSO concentration of 1% and an ionomycin final concentration of 1 μM in IP1-41 medium, and added to the cell solution. As a negative control, DMSO was adjusted to a final concentration of 1% in IP 1-41 medium and added to the cell solution. Bright-field images and fluorescence images (ex: 470/40 nm; em: 525/50 nm) of the cells before and after solution addition were taken (all-in-one fluorescence microscope BZ-X710, Keyence). The GCaMP6f stable expression cell line showed no increase in fluorescence when DMSO was added, whereas when ionomycin was added, stronger fluorescence than before the addition was confirmed. Therefore, it was confirmed that the expressed GCaMP6f functioned in the obtained stable expression cell line. For statistical analysis of fluorescence intensity, one-way ANOVA was performed using Prism8 (GraphPad), followed by Tukey's multiple comparison test as a post-hoc test,

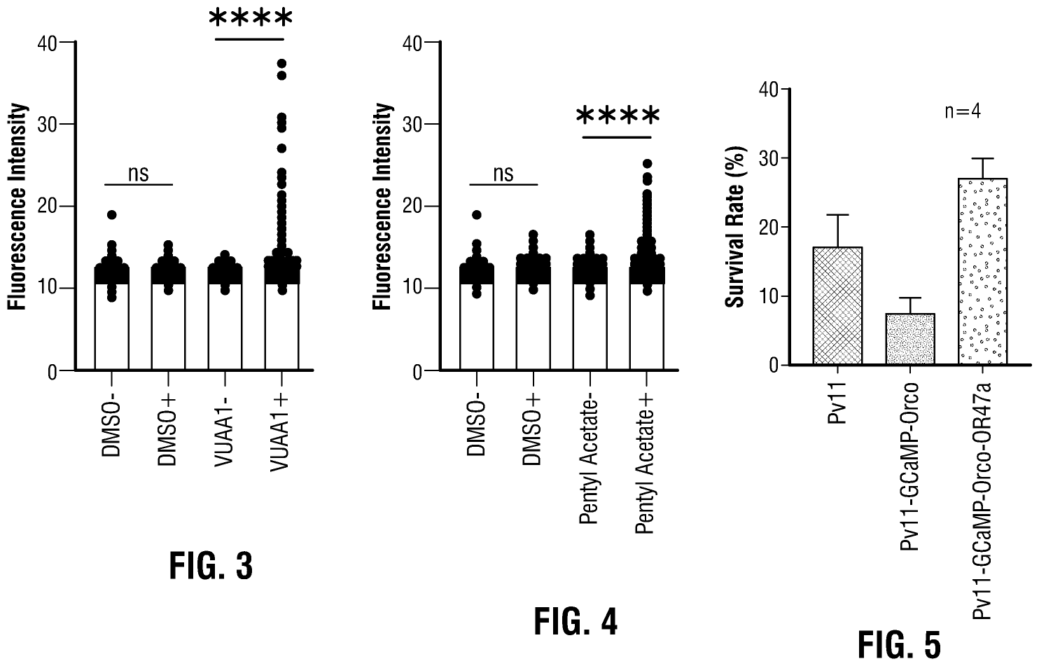

3.3 Functional confirmation of exogenous membrane proteins in cell lines with stable expression of Orco-Or47a. The amount of fluorescence expression was confirmed by the same operation as in 3.2 above. A solution adjusted to a final concentration of 100 μM of VUAA1, an Orco agonist, instead of ionomycin, was added to the cell solution. Similarly, a solution in which pentyl acetate, which is a ligand of Or47a, was adjusted to a final concentration of 1 mM, was added to the cell solution. Fluorescence images were taken at an excitation wavelength/emission wavelength of 488 nm/525 nm. The fluorescence intensity was quantified by encircling the cells in the photographed image and measuring the fluorescence intensity (F) over time. The results are shown in FIGS. 3 and 4.

As shown in FIG. 3, the Orco-Or47a stable expression cell line did not respond to a control solution containing DMSO (negative control); however, an increase in fluorescence intensity was observed for VUAA1. From this, it was found that Orco was functionally expressed. Moreover, as shown in FIG. 4, an increase in fluorescence intensity was also observed for pentyl acetate. From this, it was found that Or47a is functionally expressed. Therefore, it was found that both Orco and Or47a function normally in the prepared stable expression cell line. Statistical analysis was performed in the same manner as in 3.2 above.

4. Cell viability assay of GCaMP6f stable expression cell lines.

To assess the drought tolerance of the cell lines, the cells were dried according to the following protocol. 4×10⁷ Pv11-GCaMP6f stable expression cell lines were centrifuged at 700 g for 3 minutes and the supernatant removed. The precipitated cells were suspended in a 600 mM trehalose mix of 600 mM trehalose and IPL-41 medium at a ratio of 9:1, transferred to a 2 ml flask, and incubated at 25° C. for 48 hours. After 48 hours, the obtained cells were centrifuged at 700 g for 3 minutes and the supernatant was removed. The sedimented cells were suspended in 400 μl of 600 mM trebalose mix, and 40 μl were dropped onto a 35 mm dish. The 35 mm dish was dried for 10 days under conditions of 25° C. and humidity of 10% or less.

Cells dried for 10 days were rehydrated by adding 1 ml of IPL-41 medium. For the cell viability assay, 1.5 μl of pI solution (Dojindo Laboratories) and 4 μl of Hoechst 33342 solution (Dojindo Laboratories) were mixed in 50 μl of IPL-41 medium as a cell staining solution to prepare the staining solution MIX. 2 μl of cell staining solution MIX and 40 μl of cells after rehydration for 1 hour were placed in a 1.5 ml tube, and images were obtained by double staining. The imaging conditions were PI (ex: 545 nm/em: 605 nm) and Hoechst (ex: 405 nm/em: 460 nm). The resuscitation rate was obtained by dividing the number of viable cells (Hoechst+, p I−) in the image by the total number of cells (Hoechst+). One hour after rehydration, the control Pv11 cells had a viability of 13%, whereas the GCaMP6f stable expressing cell line had a viability of over 20%. Although genetic influences can explain differences in survival, it was demonstrated that at least the GCaMP6f stable cell line maintained drying tolerance.

5. Cell viability assay for Orco and Orco-Or47a stable cell lines. The Pv11 wild strain, Orco stable expression cell line, and Orco-Or47a stable expression cell line were dried under the conditions of 3.4 above, rehydrated, and the survival rate was confirmed 1 hour later. The results are shown in FIG. 5. It was confirmed that both the Orco stable expression cell line and the Orco-Or47a stable expression cell line-maintained drought resistance, although there was a difference in survival rate due to the effect of genetic effects.

6. Assessment of exogenous membrane protein function after drying-rehydration. Using the Orco stable expression cell line and Orco-Or47a stable expression cell line, in order to assess the function of exogenous membrane proteins before drying and after 1 hour of rehydration after drying, fluorescence intensity generated in response to stimulation with VUAA1 (Orco agonist) or pentyl acetate (ligand of Or47a) was measured in the same manner as in 3.2 above.

The fluorescence intensity was digitized from the photograph, and ΔF/F was calculated according to the following formula:

$$\frac{Δ F}{{F}_{0}} = \frac{1}{n} {\sum }_{i = 1}^{n} \frac{( {F}_{i , after} - {F}_{l , 0} )}{{F}_{l , 0}}$$

Where the summation Σᵢ₌₁ⁿ is over the n cells in the image, F_{i,after} means the fluorescence intensity of the cell i after adding the ligand solution, and F_{i,0} means the fluorescence intensity of the cell i before adding the ligand solution.

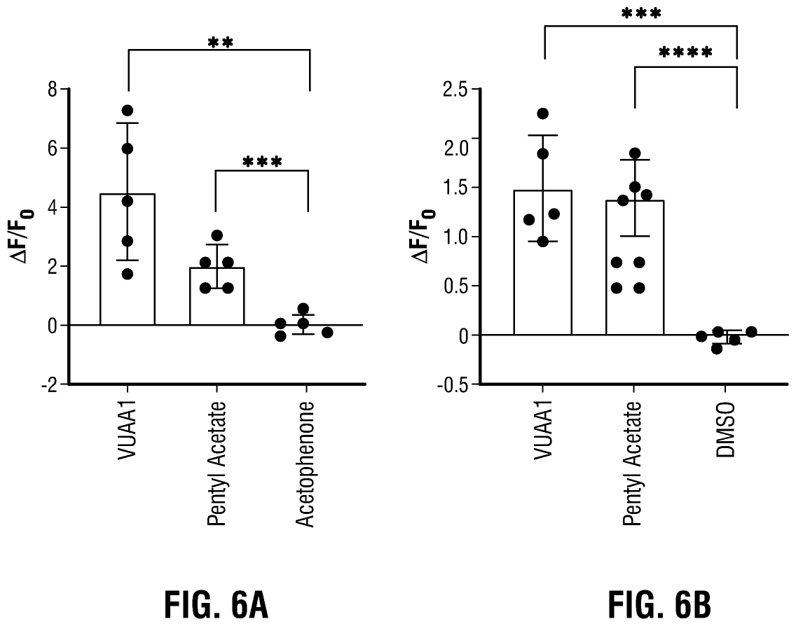

Specifically, a fluorescence image was taken under the condition in which the excitation wavelength/fluorescence wavelength was 488 nm/525 nm. In the fluorescence intensity quantification, the cells were identified in the photographed image, and the fluorescence intensity (F) was measured for each time. The initial values of fluorescence intensity (F₀) were subtracted from the measured fluorescence intensity at each time to obtain the amount of change in fluorescence (ΔF), which was then normalized by dividing by the initial value of fluorescence intensity (F₀) to obtain (Δ F/F₀). A number of cells n were selected from the captured image, ΔF/F₀ were produced for each, and the average value (including standard deviation, SD) of the results was graphed. For example, 25 cells were selected from the captured image, ΔF/F₀ for each were produced, and the averaged results (including SD) were graphed. FIG. 6A shows the results of the Orco-Or47a stable expression cell line before drying, and FIG. 6B shows the results of the Orco-Or47a stable expression cell line 1 hour after drying and rehydration. The Orco-Or47a stable expression cell line before drying responded to the Orco agonist (VUAA1) and the specific ligand of Or47a, pentyl acetate. A significant difference was confirmed in that it did not react to acetophenone, which is neither a ligand for Orco nor Or47a (FIG. 6A).

In addition, after drying treatment the dried cells were rehydrated, similarly, in the Orco-Or47a stable expression cell line after 1 hour, a significant difference in responsiveness to VUAA1 and pentyl acetate was confirmed compared to DMSO as a control (FIG. 6B).

As a result, fluorescence was observed in both the Orco-Or47a stable expression cell line into which the Orco gene was introduced and the Orco stable expression cell line under VUAA1 stimulation 1 hour after drying and rehydration. In addition, one hour after drying and rehydration, fluorescence was observed in the Orco-Or47a stable expression cell line into which the Or47a gene had been introduced by pentyl acetate stimulation. Therefore, it was found that both Orco and Or47a functions were retained in rehydrated cells after drying. In this way, it was confirmed that the functions of multiple exogenous membrane proteins were maintained after rehydration, so that it can be said that various exogenous membrane proteins can be stably preserved in a dry state by using cells derived from Polypedilum vanderplanki as a host. In addition, it was found that the exogenous membrane protein can exert its function within about 1 hour after rehydration.

7. Confirmation that exogenous membrane protein responses in Orco-Or47a stable expression cell lines immediately after rehydration are not due to de novo protein synthesis. To confirm that the membrane protein response of the Orco-Or47a stable expression cell line immediately after rehydration was not due to de novo protein synthesis, cells were treated with cycloheximide (CHX, a protein synthesis inhibitor).

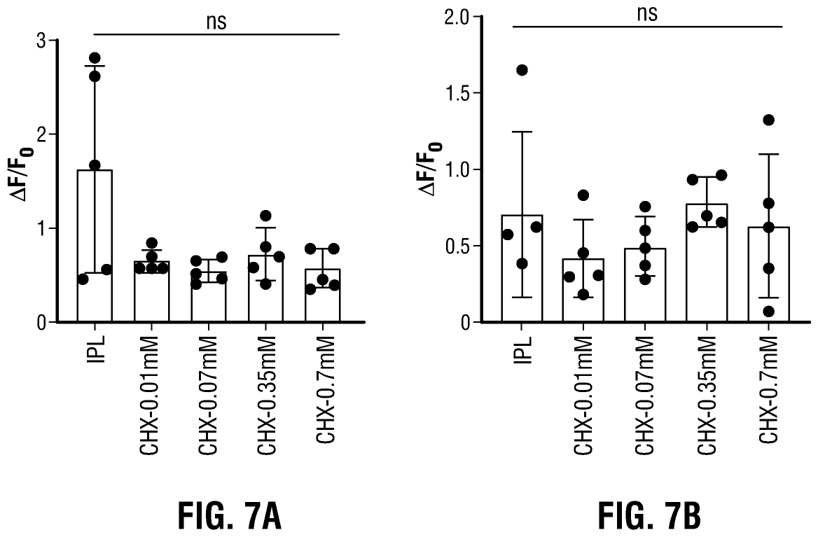

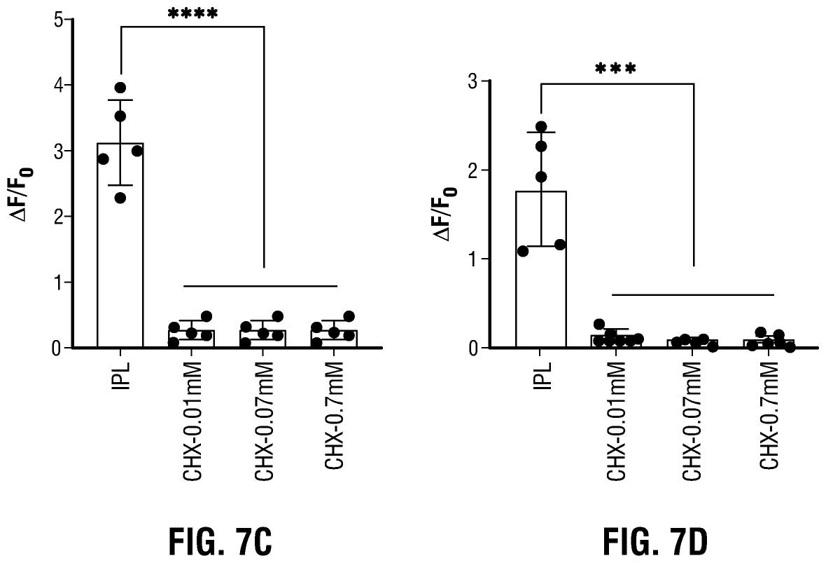

A previous study reported that 0.35 mM of cycloheximide is able to inhibit protein production in Pv11 cells. Therefore, also in this experiment, cycloheximide was dissolved in IPL-41 medium so that the cycloheximide concentration was 0.35 mM. An IPL-41 medium with 0.01 mM, 0.07 mM and 0.7 mM cycloheximide was also prepared. The rehydration of dried cells was performed using 1 ml of I PL-41 medium containing cycloheximide. One hour after rehydration and 24 hours after rehydration, the same operation as in 3.6 above was performed, and the fluorescence intensity was measured and analyzed. The ΔF/F₀ was as described above. The results are shown in FIGS. 7A-7B. FIG. 7A and FIG. 7B are graphs showing changes in fluorescence intensity of the Orco-Or47a stable expression cell line response to VUAA1 and pentyl acetate, respectively, one hour after drying and rehydration. FIGS. 7C and 7D are graphs showing changes in fluorescence intensity, respectively, in response to VUAA1 and pentyl acetate of Orco-Or47a stable expression cell lines 24 hours after drying and rehydration.

After 1 hour of drying-rehydration, Pv11 cells showed specific responses to VUAA1 (FIG. 7A) or pentyl acetate (FIG. 7B). From the results of FIGS. 7A and 7B, there was no significant difference in the function of the exogenous membrane protein depending on the presence or absence of cycloheximide and the difference in concentration. At least some of the exogenous membrane proteins Orco and Or47a were shown to function on cell membranes even after the stress of drying, storage, and rehydration. It was also confirmed that 24 hours after rehydration, the response of Pv11 cells to VUAA1 (FIG. 7C) and pentyl acetate (FIG. 7D) was strongly enhanced due to de novo synthesis of Orco and Or47a proteins. On the other hand, CHX-treated cells were less responsive to exogenous membrane proteins. As a result, Pv11 cells were found to retain Orco-Or47a function on their cell membranes to some extent even after drying and rehydration, independent of de novo protein synthesis.

8. Confirmation of behavior of fixed Pv11 cells.

8.1 Pv11 cell immobilization. A cell membrane modifier (BAM: Biocompatible Anchor for cell Membrane) was used to fix the stable expression cell line obtained above. BAM consists of a hydrophilic PEG chain, an NHS group, and a hydrophobic oleyl group; by bringing BAM into contact with a collagen-coated dish, the NHS group binds to collagen, and the oleyl group interacts with the cell membrane through hydrophobic interactions, allowing cells to adhere. BAM (SUNBRIGHT OE-040CS, Yuka Sangyo Co., Ltd.) was dissolved in DMSO (Fujifilm Wako Pure Chemical Industries) to a concentration of 10 mM. The 10 mM BAM solution was adjusted to DPBS(-) (Thermo Fisher) to a final concentration of 100 μM, 100 μl was dropped onto a collagen 1-coated 48-well microplate (IWAKI) and incubated at 37° C. for 1 hour. The BAM solution was removed, washed once with DPBS(-) (Thermo Fisher) and five times with MilliQ, and allowed to stand still to evaporate water droplets. The Pv11 cell suspension was centrifuged at 700 g for 3 minutes, the supernatant was removed, the cell pellet was suspended in DPBS(-) (Thermo Fisher) and dropped onto a BAM-coated dish. After standing for 15 minutes, the cells were washed twice with DPBS (Ca²⁺, Mg²⁺) (Thermo Fisher) to remove floating cells. The BAM fixation method confirmed that the cells could be immobilized on the plates and remained firmly attached even after the plates were washed with buffer.

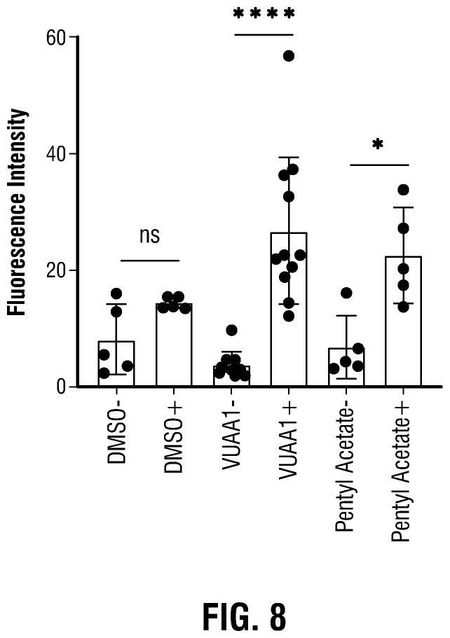

8.2 Functional confirmation of exogenous membrane proteins in fixed cells. Orco-Or47a stable expression cell lines and Orco stable expression cell lines were immobilized by the method described in 8.1 above, and their reactions to odorants were examined. Each substance was reacted with each cell in the same manner as in 3.2 above, and the fluorescence intensity was measured. The results for Pv11-GCaMP6f-Orco-Or47a stable expression cell lines are shown in FIG. 8. It was also confirmed that the immobilized Pv11-GCaMP6f-Orco-Or47a stable expression cell line responded to both the Orco agonist VUAA1 and the odorant pentyl acetate. On the other hand, the Pv11-GCaMP6f-Orco stable expression cell line did not respond to pentyl acetate but did respond to the Orco agonist VUAA1.

9. Other test examples.

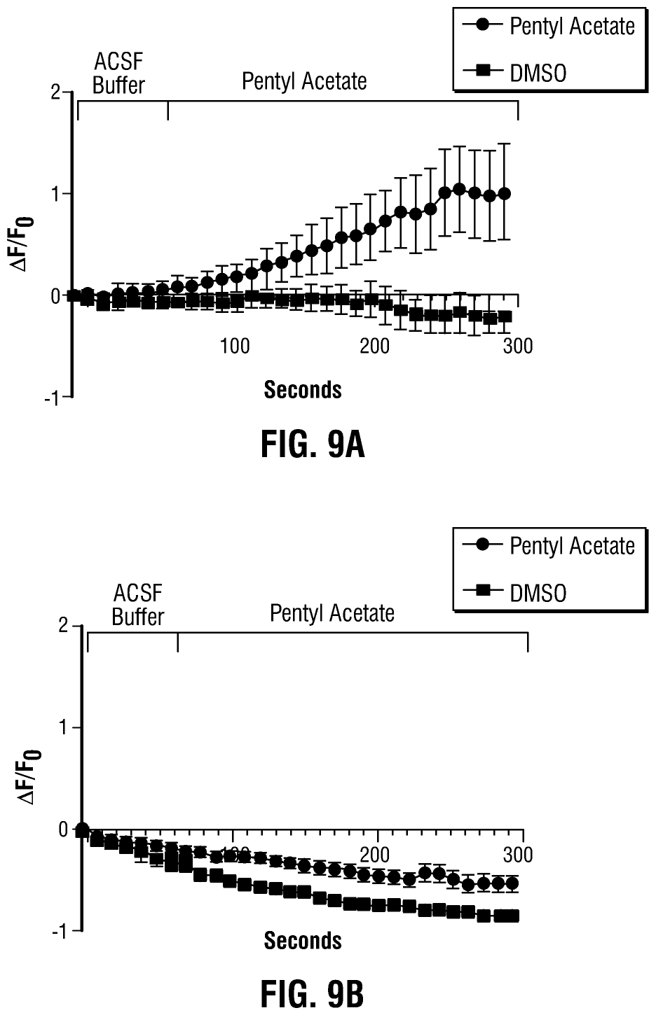

9.1 Ligand assay. A ligand assay was performed using a perfusion system using Orco-Or47a stable expression cell line and GCaMP6f stable expression cell line. FIG. 9A shows the result of the Orco-Or47a stable expression cell line, and FIG. 9B shows the result of the Pv 11-GCaMP6f stable expression cell line. The Orco-Or47a stable expression cell line and the GCaMP6f stable expression cell line were fixed with BAM as described in 8.1 above and perfused at a flow rate of 1 mL/min using a peristaltic pump (SJ-1211 II-L2, ATTO). The perfusion solution was ACSF buffer (125 mM NaCl, 5.6 mM KCl, 1.25 mM Na₂HPO₄, 10 mM HEPES, 1.5 mM CaCl₂), pH7.4) as a standard solution. As a ligand for Or47a, pentyl acetate was diluted to a final concentration of 1 mM, and as a negative control, DMSO was diluted to 1% by mass. The standard solution was perfused, switched to 1 mM pentyl acetate solution or 1% by mass DMSO solution, and perfusion was performed for 5 minutes. Photographing of the cells was started after waiting for the 1 mM pentyl acetate solution or 1% by mass DMSO solution to reach the cells in about 60 seconds. Bright-field and fluorescence images of the perfused Pv11 cells were taken 30 times at 10-second intervals (LSM700, ZEISS). The method for obtaining ΔF/F₀ was as described above. 25 cells were selected from the captured image, each produced ΔF/F₀, and the response was maintained for about 4 minutes; from this it was confirmed that the present disclosure can be applied as an odor sensor capable of rapid odor determination.

10. Example of optimization of Or47a expression

10.1 Construction of expression vector with HiBIT. An expression vector pPv121-Or47a-linker-HiBiT vector was prepared by adding a linker sequence (SEQ ID NO: 5) and a HiBiT tag to the C-terminus of the sequence encoding Or47a in the pPv121-) Or47a vector prepared in 1 above.

10.2 Addition of untranslated region (UTR). Two UTRs, Tret1 Tret1-5′UTR SEQ ID NO: 6); Tret1_3′UTR (SEQ ID NO: 7) and g5495 5′UTR (SEQ ID No: 8) and g5495_3′UTR (SEQ ID NO: 9) were cloned by the same method. By adding these UTRs to the pPv121-Orco vector prepared in 1 above and the pPv121-Or47a-linker-HiBiT, pPv121-Tret1-UTR-Orco,pPv121-g5495-UTR-Orco, pPv121-Tret1-UTR-Or47a-linker-HiBIT,pPv121-g5495-UTR-Or47a-linker-HiBiT were prepared.

10.3 Comparison of expression levels of Or47a. The expression vector and the like prepared in 6.2 above were transfected into Pv11 cells as follows to transiently express each protein.
1: Blank test (without vector introduction)
2: pPv121-Orco vector and pPv121-Or47a-linker HiBiT
3: pPv121-Tret1-UTR-Orco and pPv121-Tret1-U TR-Or47a-linker-HiBIT
4: pPv121-g5495-UTR-Orco and pPv121-g5495-U TR-Or47a-1 inker-HiBIT

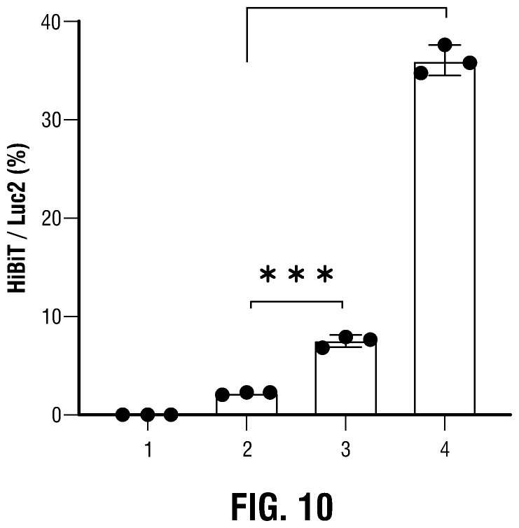

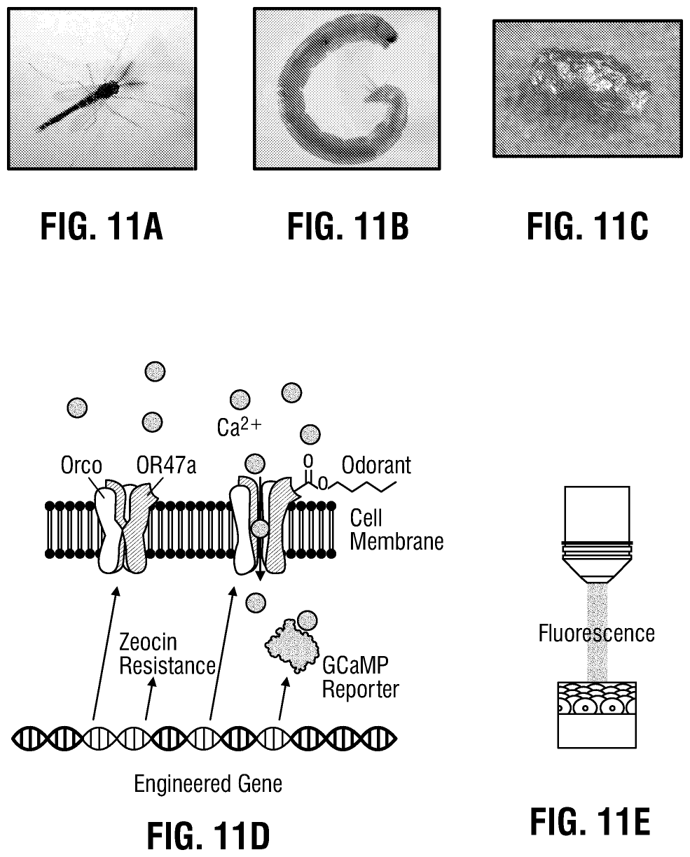

At the same time, as a transfection efficiency control, pPv121 (632 bp)-Luc2 was transfected into Pv11 cells to express Luc2. Then, for 1 to 4 above, Or47a expressed on the cell membrane was quantified using the HiBIT Extracellular detection system (Promega); separately, the expression level of Luc 2 expressed in cells was quantified using Nano-Glo (registered trademark) Dual-luciferase (registered trademark) Reporter Assay System (Promega). FIG. 10 shows the expression level of Or47a, which was corrected for transfection efficiency by dividing the Or47a value quantified by the HiBIT system by the transfection control Luc 2 value. Statistical analysis was performed in the same manner as in 3.2 above. From the results in FIG. 10, the addition of Tret1-UTR and g5495-UTR improved the expression level of Or47a Polypedilum vanderplanki is an insect dwelling in a semi-arid region (FIG. 11A) that can be completely desiccated (anhydrobiosis) in the larval state (FIG. 11B-C). As demonstrated in FIG. 11D, the Oreo/OR ion channel opens upon odorant binding. Cai influx leads to GCaMP marker fluorescence (FIG. 11E).

What is claimed:

1. Cells derived from Polypedilum vanderplanki expressing one or more exogenous membrane proteins.

2. The cells according to claim 1, wherein the one or more exogenous membrane proteins function even when the cells are dried and then rehydrated.

3. The cells according to claim 1 or 2, wherein the one or more exogenous membrane proteins comprise an olfactory receptor protein (Or) and, optionally, an olfactory receptor protein co-receptor (Orco).

4. The cells according to any one of claims 1-3, wherein the one or more exogenous membrane proteins are an Or and an Orco.

5. The cells according to any one of claims 1-4, wherein the expression of the one or more exogenous membrane proteins is controlled by a 121 promoter.

6. The cells according to any one of claims 3-5, wherein one or more additional exogenous proteins are further expressed.

7. The cells according to claim 6, wherein the one or more additional exogenous proteins comprise a fluorescent marker protein that indicates changes in intracellular calcium concentration.

8. The cell according to any one of claims 1-7, wherein the cells are Pv11 cells.

9. An odor sensor, comprising cells derived from Polypedilum vanderplanki expressing an Or and an Orco.

10. The odor sensor according to claim 9, wherein the cells derived from Polypedilum vanderplanki further express a fluorescent marker protein that indicates changes in intracellular calcium concentration.

11. The odor sensor according to claim 9 or 10, wherein the cells derived from Polypedilum vanderplanki are immobilized on a substrate.

12. An odor detection method comprising a step of detecting an odorant, or identifying an odor, using cells derived from Polypedilum vanderplanki that express an Or and an Orco.

13. An odor identification method comprising a step of detecting responses to an odorant, or identifying an odor, using a collection of cells derived from Polypedilum vanderplanki that expresses an Or and an Orco.

14. A method of preserving an exogenous membrane protein, the method comprising: (i) introducing a gene encoding the exogenous membrane protein into a cell derived from Polypedilum vanderplanki, (ii) culturing the cell to express the exogenous membrane protein in the cell membrane, and (iii) a step of drying the cells.

15. The method according to claim 14, further comprising, prior to the drying step, a step of suspending the cells in a solution comprising a dry protection agent.

16. A method of detecting, or identifying an odor, comprising the steps of (i) providing dryable insect-derived biosensor cells expressing one or more Or and an Orco, (ii) exposing the biosensor cells to at least one test compound or sample, and (iii) detecting a signal that indicates activation of the one or more OR in the dryable biosensor cells.

17. A device for detecting the presence of a particular odorant, or for identifying an odor, said device comprising dryable insect-derived biosensor cells expressing one or more ORs that are activated upon exposure to odorant molecules.

18. The device of claim 17, wherein the device is a handheld device.

19. A kit for detection of an odorant, or identification of an odor, said kit comprising one or more dryable insect-derived biosensor cells expressing one or more ORs that are activated upon exposure to odorant molecules.
